(19) United States
(12) Patent Application Publication
(10) Pub. No.: US 2024/0049965 A1
(43) Pub. Date: Feb. 15, 2024

(54) NEURAL MONITORING SYSTEM, PROCESSING APPARATUS, AND NON-TRANSITORY COMPUTER-READABLE STORAGE MEDIUM

(71) Applicant: NIHON KOHDEN CORPORATION, Tokyo (JP)

(72) Inventors: Masashi SEKIMOTO, Tokorozawa-shi (JP); Kengo EDA, Tokorozawa-shi (JP); Kazuki ICHIKAWA, Tokorozawa-shi (JP)

(73) Assignee: NIHON KOHDEN CORPORATION, Tokyo (JP)

(21) Appl. No.: 18/366,157

(22) Filed: Aug. 7, 2023

(30) Foreign Application Priority Data
Aug. 10, 2022 (JP) 2022-128151

(51) Int. Cl. A61B 5/00; A61B 5/377

(52) U.S. Cl. CPC A61B 5/0006; A61B 5/4836; A61B 5/743; A61B 5/377

(57) ABSTRACT

A neural monitoring system includes a stimulation device configured to generate a stimulation signal corresponding to stimulation applied to a subject, and a processing apparatus configured to: acquire a waveform corresponding to a change over time in an evoked potential of the subject based on the stimulation, and cause a display device to display an index indicating any one of a plurality of colors, based on an amplitude value of the waveform and an amplitude change rate. the amplitude value is acquired based on a predetermined rule. The amplitude change rate is a ratio of the amplitude value to an amplitude value of a reference waveform.

### CROSS-REFERENCE TO RELATED APPLICATIONS

This application is based on and claims priority under 35 USC 119 from Japanese Patent Application No. 2022-128151 filed on Aug. 10, 2022, the entire content of which is incorporated herein by reference.

### TECHNICAL FIELD

The present disclosure relates to a system that supports neural monitoring in a perioperative period. The present disclosure also relates to a processing apparatus included in the system and a computer program executable by a processor mounted on the processing apparatus.

### BACKGROUND ART

JP-T-2017-503620 discloses a system that supports neural monitoring in a perioperative period of a surgery. A waveform corresponding to a change over time in a motor evoked potential of a subject based on stimulation is acquired. An index indicating any one of a plurality of colors is displayed on a display device, based on an amplitude change rate of the waveform with respect to an amplitude of a reference waveform.

It is required to enhance a support performance of neural monitoring in a perioperative period.

### SUMMARY OF INVENTION

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a neural monitoring system including:
a stimulation device configured to generate a stimulation signal corresponding to stimulation applied to a subject; and
a processing apparatus configured to:
   acquire a waveform corresponding to a change over time in an evoked potential of the subject based on the stimulation; and
   cause a display device to display an index indicating any one of a plurality of colors, based on an amplitude value of the waveform and an amplitude change rate, the amplitude value being acquired based on a predetermined rule, the amplitude change rate being a ratio of the amplitude value to an amplitude value of a reference waveform.

A processing apparatus including:
an interface configured to receive a detection signal corresponding to an evoked potential of a subject; and
one or more processors configured to:
   acquire a waveform corresponding to a change over time in the evoked potential based on stimulation; and
   cause a display device to display an index indicating any one of a plurality of colors, based on an amplitude value of the waveform and an amplitude change rate, the amplitude value of the waveform acquired based on a predetermined rule, the amplitude change rate being a ratio of the amplitude value to an amplitude value of a reference waveform.

A non-transitory computer-readable storage medium storing a computer program executable by one or more processors mounted on a processing apparatus,
the computer program, when executed by the one or more processors, causing the processing apparatus to:
   acquire a waveform corresponding to a change over time in an evoked potential of a subject based on stimulation; and
   cause a display device to display an index indicating any one of a plurality of colors, based on an amplitude value of the waveform an amplitude change rate, the amplitude value acquired based on a predetermined rule, the amplitude change rate being a ratio of the amplitude value to an amplitude value of a reference waveform.

### BRIEF DESCRIPTION OF DRAWINGS

Exemplary embodiment(s) of the present invention will be described in detail based on the following figures, wherein:

FIG. 1 illustrates a functional configuration of a neural monitoring system according to an exemplary embodiment of the present invention;

FIG. 2 illustrates an example of a screen displayed on a display device of FIG. 1;

FIG. 3 illustrates correlations between an amplitude value of an evoked potential waveform, an amplitude change rate, and a color of an index;

FIG. 4 illustrates a flow of processing performed by a processing apparatus of FIG. 1;

FIG. 5 illustrates another example of the screen displayed on the display device of FIG. 1; and

FIG. 6 illustrates another example of the screen displayed on the display device of FIG. 1.

### DESCRIPTION OF EMBODIMENTS

An Embodiment will be described in detail below with reference to the accompanying drawings.

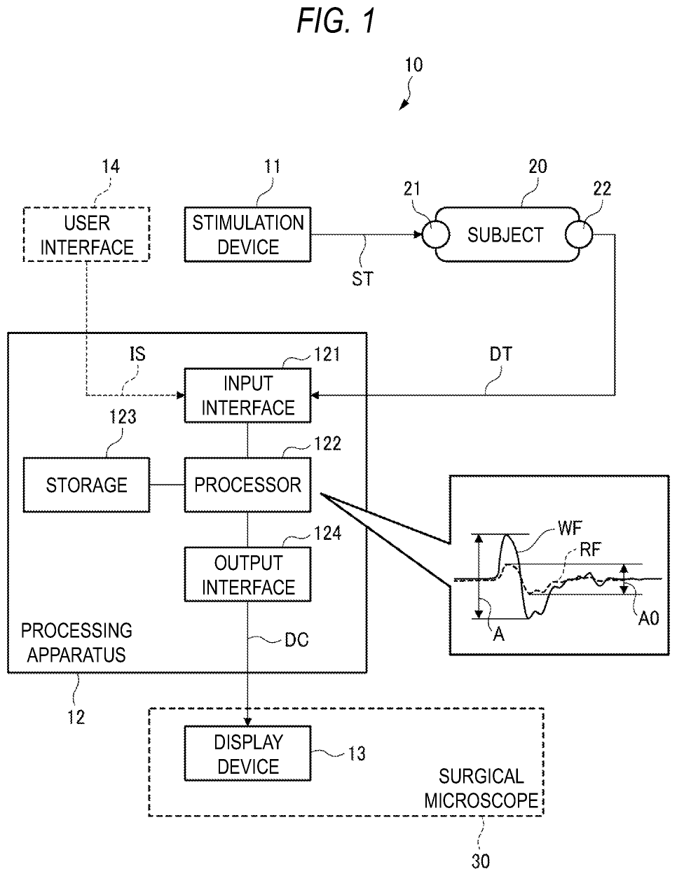

FIG. 1 illustrates a functional configuration of a neural monitoring system 10 according to an embodiment. The neural monitoring system 10 is a system for preventing paralysis caused by a neural damage by, for example, monitoring a change over time in an evoked potential of a subject 20 based on stimulation in a perioperative period of a surgery.

In the present example, a change over time in a transcranial motor evoked potential (MEP) is monitored through electrical stimulation to the subject 20.

Therefore, the neural monitoring system 10 can include a stimulation device 11. The stimulation device 11 is configured to generate a stimulation signal ST corresponding to the electrical stimulation applied through an electrode 21 attached to the subject 20. Although a single electrode 21 is illustrated in FIG. 1, a plurality of electrodes 21 are actually attached to a plurality of body parts determined according to the evoked potential to be monitored.

The MEP of the subject 20 obtained as a response to the electrical stimulation is detected through an electromyogram electrode 22 attached to the subject 20. That is, the electromyogram electrode 22 is configured to output a detection signal DT corresponding to the MEP. The detection signal DT may be an analog signal or a digital signal. Although a single electromyogram electrode 22 is illustrated in FIG. 1, a plurality of electromyogram electrodes 22 are actually attached to a plurality of body parts determined according to the evoked potential to be monitored.

The neural monitoring system 10 can include a processing apparatus 12. The processing apparatus 12 can include an input interface 121. The input interface 121 is implemented as a hardware interface that is configured to receive the detection signal DT. In a case where the detection signal DT is an analog signal, the input interface 121 can include an appropriate conversion circuit including an A/D converter.

The processing apparatus 12 can include a processor 122. The processor 122 can include one or more processors. The processor 122 is configured to acquire a waveform WF corresponding to the change over time in the MEP of the subject 20 with respect to the electrical stimulation, based on the detection signal DT received by the input interface 121.

In addition, the processor 122 is configured to acquire an amplitude value A defined as a difference between a maximum value and a minimum value of the MEP in the waveform WF. The amplitude value A is an example of an amplitude value of a waveform acquired based on a predetermined rule.

The processing apparatus 12 can include a storage 123. The storage 123 is a storage device that can be implemented by a semiconductor memory, a hard disk device, a magnetic tape device, or the like. The storage 123 is configured to store data corresponding to a reference waveform RF, which is baseline waveform. The reference waveform RF corresponds to a change over time in the MEP of the subject 20 detected under a predetermined condition before the surgery or the like. A reference amplitude value A0 is defined for the reference waveform RF. That is, the reference amplitude value A0 is defined as a difference between a maximum value and a minimum value of the MEP in the reference waveform RF.

The processor 122 is configured to acquire an amplitude change rate P that is a ratio of the amplitude value A to the reference amplitude value A0, based on the amplitude value A acquired as described above and based on the reference amplitude value A0 stored in the storage 123. In the present example, the amplitude change rate P is given by the following equation.

P=(A/A0)×100[%]

The neural monitoring system 10 can include a display device 13. The display device 13 has a configuration configured to visually provide information to a user. Examples of the configuration include a display, an indicator, and the like. The processing apparatus 12 and the display device 13 may be a part of the same device or may be devices independent of each other.

The processing apparatus 12 can include an output interface 124. The processor 122 is configured to cause the output interface 124 to output a display control signal DC, based on the amplitude value A and the amplitude change rate P acquired as described above. The display control signal DC is configured to cause the display device 13 to display an index indicating any one of a plurality of colors. The display control signal DC may be a digital signal or an analog signal, according to a specification of the display device 13. The output interface 124 is implemented as a hardware interface. In a case where the display control signal DC is an analog signal, the output interface 124 can include an appropriate conversion circuit including a D/A converter.

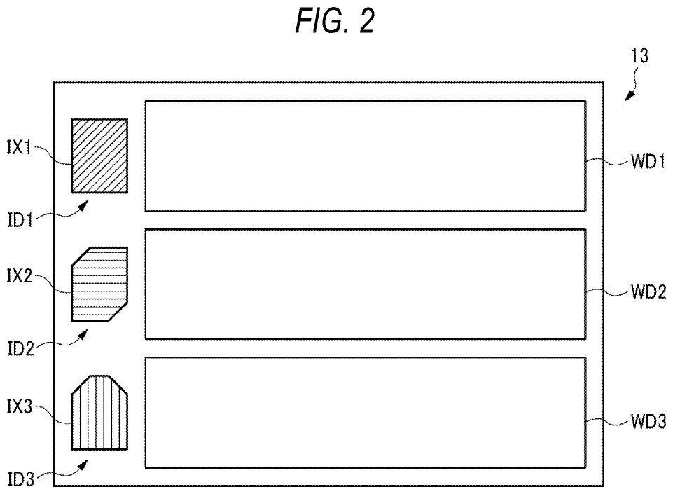

FIG. 2 illustrates an example of a screen displayed on the display device 13 based on the display control signal DC. The screen according to the present example can include a first waveform display region WD1, a second waveform display region WD2, and a third waveform display region WD3.

The first waveform display region WD1 is configured to display a first channel waveform corresponding to a change over time, in the MEP of the subject 20, related to a first channel associated with one of the plurality of electromyogram electrodes 22. In FIG. 2, illustration of the first channel waveform is omitted.

The second waveform display region WD2 is configured to display a second channel waveform corresponding to a change over time, in the MEP of the subject 20, related to a second channel associated with another one of the plurality of electromyogram electrodes 22. In FIG. 2, illustration of the second channel waveform is omitted.

The third waveform display region WD3 is configured to display a third channel waveform corresponding to a change over time, in the MEP of the subject 20, related to a third channel associated with still another one of the plurality of electromyogram electrodes 22. In FIG. 2, illustration of the third channel waveform is omitted.

The display device 13 can further include a first index display region ID1, a second index display region ID2, and a third index display region ID3.

The first index display region ID1 is configured to display a first index IX1. The first index IX1 indicates a color determined based on comparison between the first channel waveform and the reference waveform RF. The first index display region ID1 is disposed adjacent to the first waveform display region WD1. In other words, the first index display region ID1 is disposed such that the user can recognize association between the first index IX1 and the first channel waveform.

The second index display region ID2 is configured to display a second index IX2. The second index IX2 indicates a color determined based on comparison between the second channel waveform and the reference waveform RF. The second index display region ID2 is disposed adjacent to the second waveform display region WD2. In other words, the second index display region ID2 is disposed such that the user can recognize association between the second index IX2 and the second channel waveform.

The third index display region ID3 is configured to display a third index IX3. The third index IX3 indicates a color determined based on comparison between the third channel waveform and the reference waveform RF. The third index display region ID3 is disposed adjacent to the third waveform display region WD3. In other words, the third index display region ID3 is disposed such that the user can recognize association between the third index IX3 and the third channel waveform.

That is, the processing apparatus 12 is configured to cause the output interface 124 to output the display control signal DC configured to cause the display device 13 to display the first index IX1, the second index IX2, and the third index IX3 together with the first channel waveform, the second channel waveform, and the third channel waveform. The number of channels (that is, the number of indexes) of the waveform displayed on the display device 13 may be appropriately determined according to the specification of the display device 13.

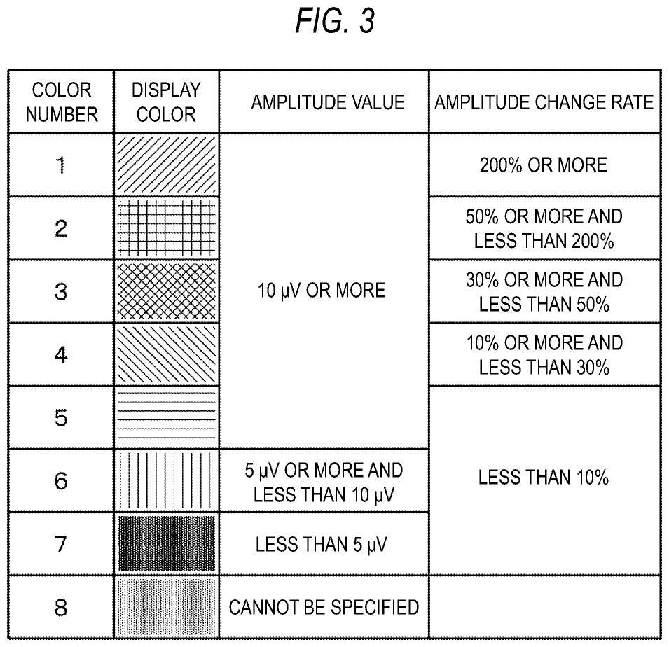

In the present example, each of the first index IX1, the second index IX2, and the third index IX3 indicates one of eight colors, according to a predetermined correlation for combinations of the amplitude value A and the amplitude change rate P. FIG. 3 illustrates correlations between the combinations of the amplitude value A and the amplitude change rate P, and colors of the index to be displayed. Data describing the correlations is stored in the storage 123 of the processing apparatus 12.

For color numbers 1 to 7 in the example illustrated in FIG. 3, the larger the number is, the higher the probability that an exercise function of the subject 20 is reduced is. Therefore, as the number increases, it is preferable to associate a display color with a stronger warning property. For example, as the number increases, the display color may be determined so as to shift from a blue-green color to a yellow-red color.

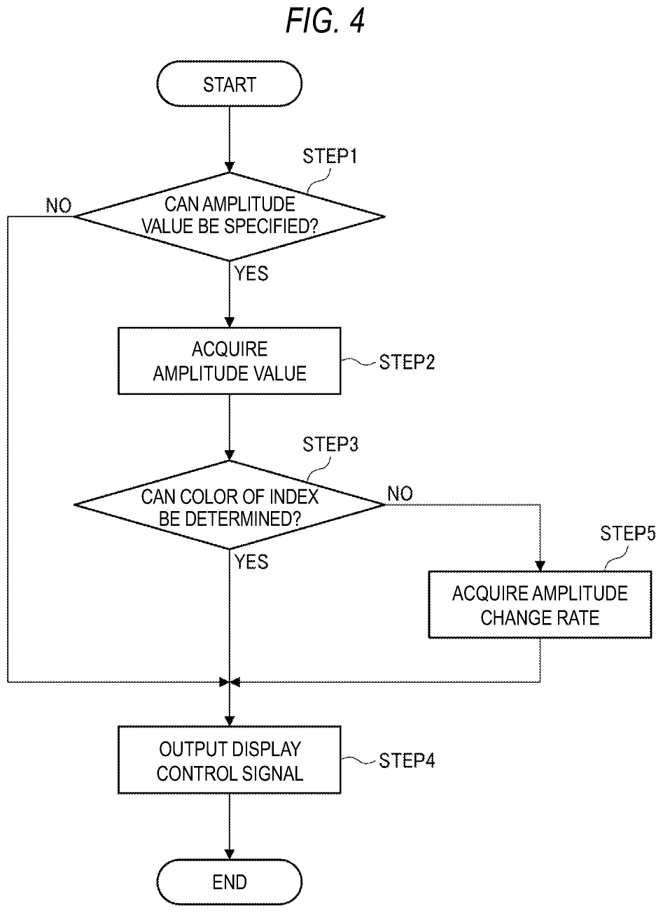

FIG. 4 illustrates a flow of processing performed by the processor 122 of the processing apparatus 12. In step 1, the processor 122 determines whether the amplitude value A of the waveform WF can be specified, based on the detection signal DT received by the input interface 121. Examples of a case where the amplitude value A is not able to be specified include the following cases. A case where the waveform WF is substantially flat and a maximum value, a minimum value, a maximal value, a minimal value, and the like of the MEP, which are necessary for specifying the amplitude value A is not able to be defined. A case where the MPF takes the maximal value and then gradually decreases to zero and the minimal value is not able to be acquired even though the maximal value and the minimal value are necessary for specifying the amplitude value A, and the like.

In a case where the processor 122 determines that the amplitude value A of the waveform WF can be specified (YES in STEP 1), the processor 122 acquires the amplitude value A of the waveform WF based on a predetermined rule (STEP 2).

Subsequently, the processor 122 determines whether the display color of the index can be determined based on the amplitude value A acquired in STEP 2 (STEP 3). In the correlation illustrated in FIG. 3, in a case where the amplitude value A is 5 μV or more and less than 10 μV and in a case where the amplitude value A is less than 5 μV, the display color of the index can be determined. In the former case in which the amplitude value A is 5 μV or more and less than 10 μV, the color number 6 is selected. In the latter case in which the amplitude value A is less than 5 μV, the color number 7 is selected. In a case where the amplitude value A is 10 μV or more, the display color of the index is not able to be determined based on only the amplitude value A.

In a case where the display color of the index is determined (YES in STEP 3), the processor 122 is configured to cause the output interface 124 to output the display control signal DC configured to cause the display device 13 to display the index indicating the color (STEP 4).

In a case where the display color of the index is not able to be determined based on only the amplitude value A (NO in STEP 3), the processor 122 acquires the amplitude change rate P, of the waveform WF, with respect to the reference waveform RF (STEP 5). Any one of the color numbers 1 to 5 is selected based on an acquired value of the amplitude change rate P and the correlation illustrated in FIG. 3. The processor 122 is configured to cause the output interface 124 to output the display control signal DC configured to cause the display device 13 to display an index indicating a color of the selected color number (STEP 4).

In the example illustrated in FIG. 2, the color number 1 is selected as a display color of the first index IX1. The color number 5 is selected as a display color of the second index IX2. The color number 7 is selected as a display color of the third index IX3.

In a case where the processor 122 determines that the amplitude value A of the waveform WF is not able to be specified (NO in STEP 1), the processor 122 is configured to cause the output interface 124 to output the display control signal DC configured to cause the display device 13 to display an index indicating the color of the color number 8 illustrated in FIG. 3 (STEP 4).

According to the configuration described above, the amplitude value A of the waveform WF is introduced as an evaluation item of the MEP in addition to the amplitude change rate P of the waveform WF with respect to the reference waveform RF. Therefore, a more subdivided evaluation related to a decrease in an exercise function of the subject 20 can be provided. For example, in the correlation illustrated in FIG. 3, a situation in which the amplitude change rate P is less than 10% is divided into three stages based on the amplitude value A. Since a result of the evaluation based on the combination of the two items is provided to the user through the color of the index, the situation can be grasped more quickly and more intuitively than based on the waveform or a plurality of numerical values. Accordingly, a support performance of exercise function monitoring in the perioperative period can be enhanced.

As described with reference to FIG. 4, in the present embodiment, first, the display color of the index is selected based on the newly introduced amplitude value A. In other words, the display color of the index is selected by giving a priority to the amplitude value A over the amplitude change rate P.

According to such a configuration, an increase in a calculation load and a processing time by the processor 122 of the processing apparatus 12 can be prevented, in a situation where an influence of the amplitude value A on the evaluation of the decrease of the exercise function of the subject 20 is relatively high. Accordingly, the support performance of the evaluation related to the decrease in the exercise function can be enhanced.

As illustrated in FIG. 2, the index displayed on the display device 13 has a rectangular shape. The rectangular shape is an example of a graphic. The processor 122 is configured to change a shape of the index to be displayed, in a case where the reference amplitude value A0 of the reference waveform RF is lower than a threshold. Specifically, the processor 122 is configured to cause the output interface 124 to output the display control signal DC that changes the shape of the index to have a shape in which an upper left corner of the rectangle shape is cut out. In the example illustrated in FIG. 2, shapes of the second index IX2 and the third index IX3 are changed as described above.

In a case where the reference amplitude value A0 of the reference waveform RF is small to some extent, the amplitude value A of the waveform WF also tends to be small. Therefore, it is necessary to pay attention to the evaluation result based on the amplitude value A. The change in the shape of the index as described above can be easily visually recognized by the user, and thus the user can be warned of the evaluation result. Therefore, the support performance of the exercise function monitoring can be further enhanced.

As illustrated in FIG. 1, the neural monitoring system 10 may include a user interface 14. The user interface 14 is configured to receive, from the user, an instruction of changing the color of the index displayed on the display device 13. The evaluation related to the decrease in an exercise ability by the processing apparatus 12 may not conform to an actual situation of the subject 20. The display color of the index may be changed later in order to record a more appropriate evaluation result in such a case.

The user interface 14 may be implemented by a switch or the like that can be mechanically operated, or may be implemented by a GUI that can be operated through an input device such as a mouse or a keyboard or through a touch panel operation. Alternatively, the instruction may be input through a voice or a gesture of the user.

The user interface 14 is configured to output an instruction signal IS corresponding to the received instruction. The instruction signal IS may be an analog signal or a digital signal, according to a specification of the user interface 14. The instruction signal IS received by the input interface 121 of the processing apparatus 12. In a case where the instruction signal IS is an analog signal, the input interface 121 can include an appropriate conversion circuit including an A/D converter.

The processor 122 is configured to change the shape of the index to be displayed in a case where the instruction signal IS corresponding to the instruction of changing the display color of the index is received by the input interface 121. Specifically, the processor 122 is configured to cause the output interface 124 to output the display control signal DC that changes the shape of the index to have a shape in which an upper right corner of the rectangle shape is cut out. In the example illustrated in FIG. 2, the shape of the third index IX3 is changed as described above.

In a case where the display color of the index is changed afterward by a certain user, another user who views the waveform WF and the index indicating a changed color may feel a sense of discomfort. Therefore, it is preferable that a fact that the display color of the index is changed by a certain user is known to another user. The change in the shape of the index as described above may be easily visually recognized by another user, and therefore another user can be prompted to recognize that the display color of the index is changed. Therefore, the support performance of the exercise function monitoring can be further enhanced.

In addition to or instead of the configuration described above, the user interface 14 may be configured to receive an instruction of changing the threshold related to the reference amplitude value A0 from the user and configured to output the instruction signal IS corresponding to the instruction.

The processor 122 may be configured to change the shape of the index to be displayed in a case where the instruction signal IS corresponding to the instruction of changing the threshold related to the reference amplitude value A0 is received by the input interface 121. Specifically, the processor 122 is configured to cause the output interface 124 to output the display control signal DC that changes the shape of the index such that the lower right corner of the rectangle shape is cut out. In the example illustrated in FIG. 2, the shape of the second index IX2 is changed as described above.

The change in the threshold related to the reference amplitude value A0 may affect a result of whether the shape of the index is changed. Therefore, it is preferable that a fact that the threshold is changed is known to the user. The change in the shape of the index as described above can also be easily visually recognized by the user, and therefore the user can be prompted to recognize that the threshold is changed. Therefore, the support performance of the exercise function monitoring can be further enhanced.

In addition to or instead of the configuration described above, the user interface 14 may be configured to receive, from the user, the instruction of changing each of the thresholds related to the amplitude value A, the amplitude change rate P, and the display color of the index illustrated in FIG. 3, and configured to output the instruction signal IS corresponding to the instruction.

In this case, the processor 122 is configured to change the shape of the index to be displayed in a case where the instruction signal IS corresponding to the instruction of changing the threshold related to the display color of the index and at least one of the amplitude value A and the amplitude change rate P is received by the input interface 121. Specifically, the processor 122 is configured to cause the output interface 124 to output the display control signal DC that changes the shape of the index such that the lower right corner of the rectangle shape is cut out. In the example illustrated in FIG. 2, the shape of the second index IX2 is changed as described above.

The change in the threshold related to the display color of the index and at least one of the amplitude value A and the amplitude change rate P may affect the evaluation result related to the exercise function of the subject 20. Therefore, it is preferable that a fact that the threshold is changed is known to the user. The change in the shape of the index as described above can also be easily visually recognized by the user, and therefore the user can be prompted to recognize that the threshold is changed. Therefore, the support performance of the exercise function monitoring can be further enhanced.

A change mode of the index shape based on the instruction of changing the threshold related to the display color of the index and at least one of the amplitude value A and the amplitude change rate P may be different from a change mode of the index shape based on the instruction of changing the threshold related to the reference amplitude value A0 of the reference waveform RF. For example, in a case where the instruction signal IS corresponding to the instruction of changing the threshold related to the amplitude change rate P is received by the input interface 121, the processor 122 may cause the output interface 124 to output the display control signal DC that changes the shape of the index such that the lower left corner of the rectangle shape is cut out.

Any method may be adopted for changing the shape of the index displayed on the display device 13. As described above, not only a change of removing a part of a graphic in an initial state but also a change of adding another graphic to the graphic in the initial state may be made. Alternatively, the rectangle shape may be changed to a graphic having a different name, such as a circle, a triangle, or a starry shape.

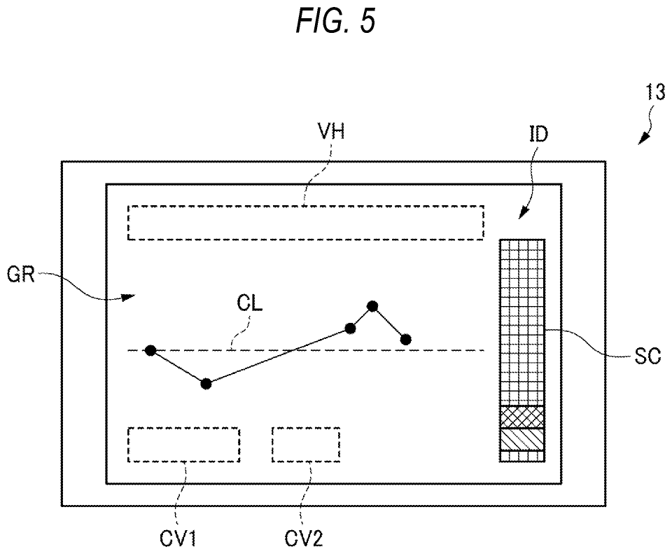

FIG. 5 illustrates another example of the screen displayed on the display device 13 based on the display control signal DC output from the processing apparatus 12. The screen according to the present example can include a graph display region GR, a first current value display region CV1, a second current value display region CV2, a value history display region VH, and an index display region ID.

The graph display region GR is configured to display, in a so-called trend form, a change over time of the amplitude change rate P acquired based on the waveform WF. A left-right direction in the graph display region GR corresponds to passage of time. Specifically, as it goes to a right direction, it corresponds to the more recent past. An up-down direction in the graph display region GR corresponds to a value of the amplitude change rate P. Specifically, an up direction corresponds to a larger value. A center line CL represents P=100%.

Every time the amplitude change rate P is acquired, plotting is performed at a position corresponding to an acquisition time point in the left-right direction of the graph display region GR. The newer amplitude change rate P is plotted to a right of the older amplitude change rate P. A graph is formed by connecting plots adjacent to each other in the left-right direction by a connection line. In a case where a current time point reaches a right end of the graph display region GR, the entire graph moves leftward and the plotting is continued.

The first current value display region CV1 is configured to display a numerical value of the amplitude change rate P corresponding to the latest plot. The second current value display region CV2 is configured to display a numerical value of the amplitude value A at a time point when the latest amplitude change rate P is acquired. The value history display region VH is configured to display, above each plot in the graph display region GR, a numerical value of the amplitude change rate P corresponding to the plot.

The index display region ID is configured to display, as the index, a scale SC including a plurality of colors based on the correlation illustrated in FIG. 3. In the example illustrated in FIG. 5, the scale SC, including the color numbers 2 to 4, corresponding to the value of the amplitude change rate P displayed in the graph display region GR, is displayed.

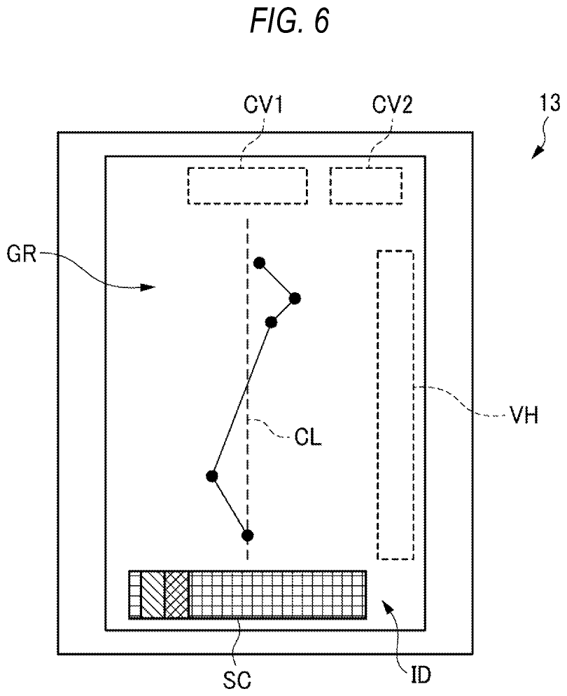

FIG. 6 illustrates another example of the screen displayed on the display device 13 based on the display control signal DC output from the processing apparatus 12. Substantially the same elements as those in the example illustrated in FIG. 5 are denoted by the same reference characters, and repeated description thereof is omitted.

The graph display region GR according to the present example is configured to display, in a so-called water-fall form, the change over time of the amplitude change rate P acquired based on the waveform WF. The up-down direction in the graph display region GR corresponds to the passage of time. Specifically, as it goes to the up direction, it corresponds to the more recent past. The left-right direction in the graph display region GR corresponds to the value of the amplitude change rate P. Specifically, the right direction corresponds to a larger value. A center line CL represents P=100%.

Every time the amplitude change rate P is acquired, the plotting is performed at an upper end of the graph display region GR. The newer amplitude change rate P is plotted above the older amplitude change rate P. A graph is formed by connecting plots adjacent to each other in the up-down direction by a connection line. While the entire graph moves downward, the plotting is continued.

The value history display region VH is configured to display a numerical value of the amplitude change rate P corresponding to the plot on the right side of each plot in the graph display region GR.

According to the screen display illustrated in FIG. 5 or FIG. 6, the user can visually grasp, through the color of the index, the change over time in the evaluation result related to the exercise function of the subject 20 based on the combination of the amplitude value A and the amplitude change rate P. Since a transition of a condition of the subject 20 until the latest evaluation result is reached can be grasped, the support performance of the exercise function monitoring can be further enhanced.

In the graph display region GR, a graph indicating a change over time in the amplitude value A may be displayed. The center line CL may represent, for example, A=10 μV. In this case, the first current value display region CV1 is configured to display the numerical value of the amplitude value A corresponding to the latest plot. The second current value display region CV2 is configured to display the numerical value of the amplitude change rate P at the time point when the latest amplitude value A is acquired. The value history display region VH is configured to display the numerical value of the amplitude value A corresponding to each plot in the graph display region GR.

The processor 122 of the processing apparatus 12 having various functions described above may be implemented by a general-purpose microprocessor that operates in cooperation with a general-purpose memory. Examples of the general-purpose microprocessor include a CPU, an MPU, and a GPU. Examples of the general-purpose memory include a ROM and a RAM. In this case, the ROM may store a computer program for implementing the function. The ROM is an example of a non-transitory computer-readable medium that stores a computer program. The general-purpose microprocessor designates at least a part of the programs stored in the ROM, loads the programs in the RAM, and executes the above-described processing in cooperation with the RAM. The computer program may be pre-installed in the general-purpose memory, or may be downloaded from an external server via a communication network and then installed in the general-purpose memory. In this case, the external server is an example of a non-transitory computer-readable medium that stores the computer program.

The processor 122 may be implemented by a dedicated integrated circuit such as a microcontroller, an ASIC, or an FPGA that are includes a storage element in which a computer program for implementing the function is pre-installed. In this case, the storage element is an example of a non-transitory computer-readable medium in which a computer program is stored.

The processor 122 may be implemented by a combination of a general-purpose microprocessor and a dedicated integrated circuit.

The configurations described above are merely examples for facilitating understanding of the present disclosure. The configurations may be appropriately changed or combined with another configuration without departing from the gist of the present disclosure.

The amplitude value A acquired for the waveform WF does not need to be the difference between the maximum value and the minimum value of the MEP. In a case where two points that can serve as references in the waveform WF are specified based on a predetermined rule, a difference in the MEP between the two points can be acquired as the amplitude value A. Examples of such two points include the maximal value and the minimal value, the maximum value or the minimum value and a base line potential, and a maximal value or a minimal value and the base line potential.

The display device 13 may be an independent device having a display function, or may be provided as a part of another device. For example, as illustrated in FIG. 1, the display device 13 may be a part of the surgical microscope 30. In this case, the screens illustrated in FIGS. 2, 5, and 6 may be displayed in a field of view of the surgical microscope 30. According to such a configuration, determination related to a surgery can be performed while confirming the evaluation result related to the exercise function of the subject 20. An advantage of the configuration according to the present disclosure that provides the evaluation result through the color of the index, is more remarkable in a case where the configuration is applied to the surgical microscope 30 having a limited field of view.

A target of the neural monitoring is not limited to the MEP. A somatosensory evoked potential (SEP), a visual evoked potential (VEP), an auditory brainstem response (ABR), and the like can also be targets of the monitoring. In a case where the SEP is the target, the stimulation device 11 applies the electrical stimulation to the subject 20, and a sensory function of the subject 20 is evaluated. In a case where the VEP is the target, the stimulation device 11 applies optical stimulation to a retina of the subject 20, and a visual neural function of the subject 20 is evaluated. In a case where the ABR is the target, the stimulation device 11 applies sound stimulation to the subject 20, and an auditory neural function of the subject 20 is evaluated.

The configurations to be listed below also constitute a part of the present disclosure.

(1): A neural monitoring system including:
a stimulation device configured to generate a stimulation signal corresponding to stimulation applied to a subject; and
a processing apparatus configured to:
   acquire a waveform corresponding to a change over time in an evoked potential of the subject based on the stimulation; and
   cause a display device to display an index indicating any one of a plurality of colors, based on an amplitude value of the waveform and an amplitude change rate, the amplitude value being acquired based on a predetermined rule, the amplitude change rate being a ratio of the amplitude value to an amplitude value of a reference waveform.

(2): The neural monitoring system according to (1),
in which the processing apparatus is further configured to cause the display device to display the index together with the waveform.

(3): The neural monitoring system according to (1),
in which the processing apparatus is further configured to cause the display device to display the index together with a change over time in one of the amplitude value and the amplitude change rate.

(4): The neural monitoring system according to any one of (1) to (3),
in which the processing apparatus is further configured to:
   select a color of the index based on the amplitude value; and
   select the color of the index based on the amplitude change rate, in a case where the color of the index is not able to be selected based on only the amplitude value.

(5): The neural monitoring system according to any one of (1) to (4),
in which the index is a graphic, and
the processing apparatus is further configured to change a shape of the graphic in a case where the amplitude value of the reference waveform is less than a threshold.

(6): The neural monitoring system according to (5),
in which the processing apparatus is further configured to change the shape of the graphic, based on an instruction of changing the threshold.

(7): The neural monitoring system according to any one of (1) to (6),
in which the index is a graphic, and the processing apparatus is further configured to change a shape of the
graphic, based on an instruction of changing a color of the index.

(8): The neural monitoring system according to any one of (1) to (7),
in which the index is a graphic, and
the processing apparatus is further configured to change a shape of the graphic, based on an instruction of changing a correlation between a color of the index and at least one of the amplitude value and the amplitude change rate.

(9): The neural monitoring system according to any one of (1) to (8),
in which the display device is provided in a field of view of a surgical microscope.

(10): A processing apparatus including:
an interface configured to receive a detection signal corresponding to an evoked potential of a subject; and
one or more processors configured to:
   acquire a waveform corresponding to a change over time in the evoked potential based on stimulation; and
   cause a display device to display an index indicating any one of a plurality of colors, based on an amplitude value of the waveform and an amplitude change rate, the amplitude value of the waveform acquired based on a predetermined rule, the amplitude change rate being a ratio of the amplitude value to an amplitude value of a reference waveform.

(11): A non-transitory computer-readable storage medium storing a computer program executable by one or more processors mounted on a processing apparatus,
the computer program, when executed by the one or more processors, causing the processing apparatus to:
   acquire a waveform corresponding to a change over time in an evoked potential of a subject based on stimulation; and
   cause a display device to display an index indicating any one of a plurality of colors, based on an amplitude value of the waveform an amplitude change rate, the amplitude value acquired based on a predetermined rule, the amplitude change rate being a ratio of the amplitude value to an amplitude value of a reference waveform.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A neural monitoring system comprising:
a stimulation device configured to generate a stimulation signal corresponding to stimulation applied to a subject; and
a processing apparatus configured to:
acquire a waveform corresponding to a change over time in an evoked potential of the subject based on the stimulation; and
cause a display device to display an index indicating any one of a plurality of colors, based on an amplitude value of the waveform and an amplitude change rate, the amplitude value being acquired based on a predetermined rule, the amplitude change rate being a ratio of the amplitude value to an amplitude value of a reference waveform.

2. The neural monitoring system according to claim 1,
wherein the processing apparatus is further configured to cause the display device to display the index together with the waveform.

3. The neural monitoring system according to claim 1,
wherein the processing apparatus is further configured to cause the display device to display the index together with a change over time in one of the amplitude value and the amplitude change rate.

4. The neural monitoring system according to claim 1,
wherein the processing apparatus is further configured to:
select a color of the index based on the amplitude value; and
select the color of the index based on the amplitude change rate, in a case where the color of the index is not able be selected based on only the amplitude value.

5. The neural monitoring system according to claim 1,
wherein the index is a graphic, and
the processing apparatus is further configured to change a shape of the graphic, in a case where the amplitude value of the reference waveform is less than a threshold.

6. The neural monitoring system according to claim 5,
wherein the processing apparatus is further configured to change the shape of the graphic, based on an instruction of changing the threshold.

7. The neural monitoring system according to claim 1,
wherein the index is a graphic, and
the processing apparatus is further configured to change a shape of the graphic, based on an instruction of changing a color of the index.

8. The neural monitoring system according to claim 1,
wherein the index is a graphic, and
the processing apparatus is further configured to change a shape of the graphic, based on an instruction of changing a correlation between a color of the index and at least one of the amplitude value and the amplitude change rate.

9. The neural monitoring system according to claim 1,
wherein the display device is provided in a field of view of a surgical microscope.

10. A processing apparatus comprising:
an interface configured to receive a detection signal corresponding to an evoked potential of a subject; and
one or more processors configured to:
acquire a waveform corresponding to a change over time in the evoked potential based on stimulation; and
cause a display device to display an index indicating any one of a plurality of colors, based on an amplitude value of the waveform and an amplitude change rate, the amplitude value of the waveform acquired based on a predetermined rule, the amplitude change rate being a ratio of the amplitude value to an amplitude value of a reference waveform.

11. A non-transitory computer-readable storage medium storing a computer program executable by one or more processors mounted on a processing apparatus,
the computer program, when executed by the one or more processors, causing the processing apparatus to:
acquire a waveform corresponding to a change over time in an evoked potential of a subject based on stimulation; and
cause a display device to display an index indicating any one of a plurality of colors, based on an amplitude value of the waveform an amplitude change rate, the amplitude value acquired based on a predetermined rule, the amplitude change rate being a ratio of the amplitude value to an amplitude value of a reference waveform.
